SEQUENCE LISTING (24 sequences)

SEQ ID NO: 1 - DNA, 25 bp, Artificial Sequence
  Synthetic Primer
gctggaggtagtagagcctgaagtc

SEQ ID NO: 2 - DNA, 22 bp, Artificial Sequence
  Synthetic Primer
ggagtcaggctgttcaagacaa

SEQ ID NO: 3 - DNA, 34 bp, Artificial Sequence
  Synthetic Primer
tgcaggactcactggatagatgttattcaactcc

SEQ ID NO: 4 - DNA, 22 bp, Artificial Sequence
  Synthetic Primer
tgtgagcagcatgtggactaca

SEQ ID NO: 5 - DNA, 25 bp, Artificial Sequence
  Synthetic Primer
tcaggagagatggtgaattcttttg

SEQ ID NO: 6 - DNA, 33 bp, Artificial Sequence
  Synthetic Primer
aagaccatcttggaccaaggaagaaatatcctc

SEQ ID NO: 7 - DNA, 19 bp, Artificial Sequence
  Synthetic Primer
ccttccaggccagcttttg

SEQ ID NO: 8 - DNA, 24 bp, Artificial Sequence
  Synthetic Primer
cgaagcctttcagattactcatga

SEQ ID NO: 9 - DNA, 28 bp, Artificial Sequence
  Synthetic Primer
catgcttcccttctttccctctaccctg

SEQ ID NO: 10 - DNA, 22 bp, Artificial Sequence
  Synthetic Primer
gtccgggatgttggtatgaatt

SEQ ID NO: 11 - DNA, 21 bp, Artificial Sequence
  Synthetic Primer
ccaagccatttgctctgttga

SEQ ID NO: 12 - DNA, 27 bp, Artificial Sequence
  Synthetic Primer
tcctctactcctggctcagaaacgccc

SEQ ID NO: 13 - DNA, 20 bp, Artificial Sequence
  Synthetic Primer
cgaaaccacttggccttcag

SEQ ID NO: 14 - DNA, 27 bp, Artificial Sequence
  Synthetic Primer
gctttcagctttcgtaaatattcagtt

SEQ ID NO: 15 - DNA, 22 bp, Artificial Sequence
  Synthetic Primer
ccctgcctcggccagaggtttc

SEQ ID NO: 16 - DNA, 20 bp, Artificial Sequence
  Synthetic Primer
tggcttcttcctgcgaattg

SEQ ID NO: 17 - DNA, 22 bp, Artificial Sequence
  Synthetic Primer
tctgctgggctacacttctcaa

SEQ ID NO: 18 - DNA, 22 bp, Artificial Sequence
  Synthetic Primer
aaggacaccgagggccacctgg

SEQ ID NO: 19 - DNA, 20 bp, Artificial Sequence
  Synthetic Primer
aggaatctgcaggagtctga

SEQ ID NO: 20 - DNA, 20 bp, Artificial Sequence
  Synthetic Primer
tacgaggccaacatttcagg

SEQ ID NO: 21 - DNA, 20 bp, Artificial Sequence
  Synthetic Primer
agaggaggaagtcccccagg

SEQ ID NO: 22 - DNA, 18 bp, Artificial Sequence
  Synthetic Primer
gatttggacctgcgagcg

SEQ ID NO: 23 - DNA, 18 bp, Artificial Sequence
  Synthetic Primer
gcggctgtctccacaagt

SEQ ID NO: 24 - DNA, 22 bp, Artificial Sequence
  Synthetic Primer
ttctgacctgaaggctctgcgc

(19) United States
(12) Patent Application Publication
(10) Pub. No.: US 2015/0232927 A1
(43) Pub. Date: Aug. 20, 2015

(54) EVAULATION OF DUF1220 COPY NUMBER AND METHODS OF USING THE SAME

(71) Applicant: James M. SIKELA, (US)

(72) Inventor: James M. Sikela, Centennial, CO (US)

(21) Appl. No.: 14/422,083

(22) PCT Filed: Aug. 15, 2013
(86) PCT No.: PCT/US13/55194

§ 371 Date: Feb. 17, 2015

Related U.S. Application Data
(60) Provisional application No. 61/683,632, filed on Aug. 15, 2012.

(51) Int. Cl. C12Q 1/68; A61K 38/17

(52) U.S. Cl. CPC C12Q 1/6876; A61K 38/17; C12Q 2600/124; C12Q 2600/118; C12Q 2600/16

(57) ABSTRACT

Methods of selecting individuals having or predicted to develop abnormalities in brain volume, that may include microcephaly, or macrocephaly, and may manifest in neurological disorders such as schizophrenia or autism. Methods of selecting individuals having or predicted to develop low or high cognitive function, such as low or high IQ. Therapeutic methods of delivering DUF1220 domain protein products or fragments or mimetics thereof to enhance cognition in an individual, including patients with cognitive disorders, dementia, neurodegenerative disorders, and the like.

### CROSS REFERENCE TO RELATED APPLICATION

This application claims the benefit of priority under 35 U.S.C. §119(e) to U.S. Provisional Patent Application Ser. No. 61/683,632 filed Aug. 15, 2012, which is incorporated herein by reference.

### TECHNICAL FIELD

The invention relates to methods of evaluating protein domain copy number and the diagnostic and prognostic subject evaluations that are made in view of this determination.

### BACKGROUND OF THE DISCLOSURE

### DUF1220 Domain

DUF1220 is a protein domain of unknown function that shows a striking human lineage-specific (HLS) increase in copy number and is associated with human brain evolution. DUF1220 domains are approximately 65 amino acids in length and are encoded by a two-exon doublet. In the human genome, DUF1220 sequences are located primarily on chromosome 1 in region 1q21.1-q21.2, with several copies also found at 1p36, 1p13.3, and 1p12. Sequences encoding DUF1220 domains show signs of positive selection, especially in primates, and are expressed in several human tissues including brain, where their expression is restricted to neurons.

The copy number of the DUF1220 domain increases as a function of a species evolutionary proximity to humans. DUF1220 copy number is highest in humans, having over 270 copies, with person-to-person variations in copy number, and shows the largest HLS increase in copy number (an additional 160 copies) of any protein coding region in the human genome. DUF1220 copy number is reduced in African great apes (estimated 125 copies in chimpanzees), further reduced in orangutan (92) and Old World monkeys (35), single- or low-copy in non-primate mammals and absent in non-mammals.

### Microcephaly

Microcephaly is a neurodevelopmental disorder in which the circumference of the head is more than two standard deviations smaller than average for the person's age and sex. Microcephaly may be congenital or it may develop in the first few years of life. The disorder may stem from a wide variety of conditions that cause abnormal growth of the brain, or from syndromes associated with chromosomal abnormalities. Two copies of a loss-of-function mutation in one of the microcephalin genes causes primary microcephaly. Several additional genes have been identified that cause primary microcephaly, but there are many cases of microcephaly for which the underlying genetic cause is not yet known. In general, life expectancy for individuals with microcephaly is reduced and the prognosis for normal brain function is poor. The prognosis varies depending on the presence of associated abnormalities.

Affected newborns generally have striking neurological defects and seizures. Severely impaired intellectual development is common, but disturbances in motor functions may not appear until later in life.

Infants with microcephaly are born with either a normal or reduced head size. Subsequently the head fails to grow while the face continues to develop at a normal rate, producing a child with a small head and a receding forehead, and a loose, often wrinkled scalp. As the child grows older, the smallness of the skull becomes more obvious, although the entire body also is often underweight and dwarfed. Development of motor functions and speech may be delayed. Hyperactivity and mental retardation are common occurrences, although the degree of each varies. Convulsions may also occur. Motor ability varies, ranging from clumsiness in some to spastic quadriplegia in others. Genetic relationships have been found between schizophrenia, deletions of chromosomes and microcephaly. Generally there is no specific treatment for microcephaly; treatment is symptomatic and supportive.

### Macrocephaly

Macrocephaly occurs when the head is abnormally large; this includes the scalp, the cranial bone, and the contents of the cranium. Macrocephaly may be pathologic, but many people with an unusually large head are healthy. Pathologic macrocephaly may be due to megalencephaly (enlarged brain), hydrocephalus (water on the brain), cranial hyperostosis (bone overgrowth), and other conditions. Pathologic macrocephaly is called “syndromic” when it is associated with any other noteworthy condition, and “non-syndromic” otherwise. Pathologic macrocephaly can be caused by congenital anatomic abnormalities, genetic conditions or by environmental events.

Many genetic conditions are associated with macrocephaly, including familial macrocephaly, autism, PTEN mutations such as Cowden disease, neurofibromatosis type 1, and tuberous sclerosis; overgrowth syndromes such as Sotos syndrome (cerebral gigantism), Weaver syndrome, Simpson-Golabi-Behmel syndrome (Bulldog syndrome), and macrocephaly-capillary malformation (M-CMTC) syndrome; neuro-cardio-facial-cutaneous syndromes such as Noonan syndrome, Costello syndrome, and cardiofaciocutaneous syndrome; Fragile X syndrome; leukodystrophies (brain white matter degeneration) such as Alexander disease, Canavan disease, and megalencephalic leukoencephalopathy with subcortical cysts; and glutaric aciduria type 1 and D-2-hydroxyglutaric aciduria.

Genetic relations have been found between autism, duplications of segments of chromosomes and macrocephaly on one side.

Environmental events associated with macrocephaly include infection, neonatal intraventricular hemorrhage (bleeding within the infant brain), subdural hematoma (bleeding beneath the outer lining of the brain), subdural effusion (collection of fluid beneath the outer lining of the brain), and arachnoid cysts (cysts on the brain surface).

Macrocephaly is customarily diagnosed if head circumference is greater than 2 standard deviations (SD) above the mean. Relative macrocephaly occurs if the measure is less than 2 SD above the mean but is disproportionately above that when ethnicity and stature are considered. Cranial height or brain imaging may also be used to determine intracranial volume more accurately.

### Cognition

Cognition can be defined as the processes an organism uses to organize information. This includes acquiring information (perception), selecting (attention), representing (understanding) and retaining (memory) information, and using it to guide behavior (reasoning and coordination of motor outputs). Interventions to improve cognitive function may be directed at any one of these core faculties.

An intervention that is aimed at correcting a specific pathology or defect of a cognitive subsystem may be characterized as therapeutic. Alternatively, a cognitive enhancement is an intervention that improves a subsystem in some way other than repairing something that is broken or remedying a specific dysfunction. In practice, the distinction between therapy and enhancement is often difficult to discern, and it could be argued that it lacks practical significance. For example, cognitive enhancement of a subject whose natural memory is poor could leave that person with a memory that is still worse than that of another person who has retained a fairly good memory despite suffering from an identifiable pathology, such as early-stage Alzheimer's disease. A cognitively enhanced person, therefore, is not necessarily somebody with particularly high (let alone super-human) cognitive capacities. A cognitively enhanced person, rather, is somebody who has benefited from an intervention that improves the performance of some cognitive subsystem without correcting some specific, identifiable pathology or dysfunction of that subsystem.

The spectrum of cognitive enhancements includes not only medical interventions, but also psychological interventions (such as learned “tricks” or mental strategies), as well as improvements of external technological and institutional structures that support cognition. A distinguishing feature of cognitive enhancements, however, is that they improve core cognitive capacities rather than merely particular narrowly defined skills or domain-specific knowledge.

Most efforts to enhance cognition are of a rather mundane nature, and some have been practiced for thousands of years. The prime example is education and training, where the goal is often not only to impart specific skills or information, but also to improve general mental faculties such as concentration, memory, and critical thinking. Other forms of mental training, such as yoga, martial arts, meditation, and creativity courses are also in common use. Caffeine is widely used to improve alertness. Herbal extracts reputed to improve memory are popular, with sales of Ginko biloba alone in the order of several hundred million dollars annually in the U.S. In an ordinary supermarket there are a staggering number of energy drinks for consumers who are hoping to enhance their cognitive function. Education and training, as well as the use of external information processing devices, may be labeled as “conventional” means of enhancing cognition. They are often well established and culturally accepted. By contrast, methods of enhancing cognition through “unconventional” means, such as deliberately created nootropic drugs, gene therapy, or neural implants, are nearly all to be regarded as experimental at the present time.

### SUMMARY OF INVENTION

DUF1220 domain dosage is a key factor in the determination of primate and human brain size. DUF1220 domains are approximately 65 amino acids in length and have undergone rapid and extensive copy number expansion during recent primate evolution, most strikingly in the human lineage. The present inventor has developed high resolution assays to identify genomic sequences, and specifically DUF1220 copy number variations, associated with brain size variations in disease and nondisease populations.

The present inventor's studies regarding DUF1220 domain dosage and its correlation with certain brain characteristics throughout evolution suggests that modulation of the DUF1220 domain through increased or decreased expression, or by direct means of increasing the expression products of the DUF1220 domain, or by adding more copies of DNA sequences encoding DUF1220 domains, may enhance cognitive function in a mammal.

The present invention is generally related to the identification of individuals that are predicted to have, or to develop, a neurodevelopmental disorder, such as microcephaly or macrocephaly, (i.e., the identification of individuals at an increased risk of developing an abnormal brain size). The present invention is also generally related to methods to identify treatments that can improve the cognitive function of individuals that have or may develop an abnormal brain size. The present invention is also generally related to methods to identify individuals that may have or may develop abnormally low or high cognitive function, including low or high intelligence quotient (IQ). The present invention is also generally related to the development of adjuvant treatments that enhance cognitive function in an individual through the modulation of DUF1220 protein activity or through addition or delivery of more DUF1220 domain protein.

Accordingly, one aspect of the disclosure relates to methods, and corresponding assay kits, for use to select an individual who is predicted to develop a neurodevelopmental disorder. In certain embodiments, the neurodevelopmental disorder is microcephaly or macrocephaly. The method generally includes detecting in a biological sample from an individual the copy number of the DUF1220 protein domain that have been discovered by the inventor to be valuable in predicting the individual's development of a neurodevelopmental disorder.

Based on the inventor's discovery, a variety of tests and detection strategies are proposed, and discussed in detail below. Initially, however, the present invention includes the use of the following strategies for detection of biomarkers, alone or in various combinations: (1) detection of the level of DNA copy number or protein amplification of the (DUF1220) protein domain (i.e., DNA sequences encoding DUF1220 domains or a protein segment containing DUF1220 protein domain sequences); (2) detection of the level of DNA copy number variation for sequences encoding the DUF1220 protein domain; (3) detection of mutations in the DUF1220 domain; and, (4) detection of DUF1220 protein expression. These detection protocols may be used individually or in various combinations, and certain embodiments further include the use of various combinations of one or more biomarker detection techniques to further enhance the ability of the present method to identify individuals that may develop neurodevelopmental disorders.

The inventors have also discovered that DUF1220 copy number (i.e. the CON2 subtype of DUF1220) shows a statistically significant correlation with IQ and/or IQ-like cognitive performance scores. In other words, individuals with statistically-high or low DUF1220 copy number have high or low cognitive function, respectively. Thus, another aspect of the disclosure relates to methods, and corresponding assay kits, for use in measuring DUF1220 copy number as a means of predicting an individual's level of cognitive function or cognitive ability. In certain embodiments, the cognitive function predicted in the individual will be a high or low IQ. The method generally includes detecting in a biological sample from an individual the copy number of DNA sequences encoding the DUF1220 protein domain that has been discovered by the inventor to be valuable in predicting the individual's cognitive function or innate cognitive capacity.

The present inventor has discovered that individuals with cells having an extreme increase in DUF1220 copy number, i.e., amplification of DNA sequences encoding DUF1220 domains, with respect to the DUF1220 protein domain (also generally referred to herein as an increase in the DUF1220 domain), are predicted to be at increased risk of developing an abnormally large brain size, e.g. macrocephaly.

Additionally, the present inventor has discovered that individuals with cells having decreased DUF1220 domain copy number are predicted to be at increased risk of developing an abnormally small brain size, e.g. microcephaly.

Additionally, the present inventor has discovered that individuals with cells having increased DUF1220 domain (CON2 subtype) copy number, are predicted to have or develop higher cognitive function, including high IQ. Similarly, the present inventor has discovered that individuals with cells having decreased DUF1220 domain copy number, are predicted to have or develop lower cognitive function, including low IQ.

The present inventor has demonstrated that individuals having cells having a DUF1220 domain copy number in the upper 90% percentile of DUF1220 domain copy number for that individual's species have a high likelihood of developing macrocephaly, whereas individuals having cells having a DUF1220 domain copy number in the lower 10% percentile of DUF1220 domain copy number for that individual's species have a high likelihood of developing microcephaly.

Prior to the present invention, the prognostic role of DUF1220 protein expression or domain copy number in cognitive function has been unclear at best, as there have been varying reports of its significance (or lack thereof) in the literature. The inventor has studied the prognostic role of DUF1220 domain copy number and found that high DUF1220 domain copy number correlates with the incidence and development of macrocephaly, as well as increased cognitive function, including IQ, in the individual. Likewise, the inventor has found that low DUF1220 domain copy number correlates with the incidence and development of microcephaly as well as decreased cognitive function, including IQ, in the individual.

The distinction between increases in DUF1220 domain copy number that leads to macrocepahly (a neurodevelopmental disorder with harmful or disadvantageous neurological phenotype), and increased DUF1220 domain copy number leading to higher cognitive function (which is not a disorder, but instead provides a cognitive gain or benefit) is understood. In the case of macrocephaly, additional genes adjacent to DUF1220 domain sequences are increased in copy number along with DUF1220. For improved cognition, DUF1220 copy number increase typically occurs without a change in copy number of adjacent genes.

The methods and test kits provided by the present invention are useful for selecting individuals that may develop microcephaly, macrocephaly, learning disabilities, cognitive dysfunction, neurodevelopmental disorders, or neurological disorders (including schizophrenia or autism). Such individuals might, as a result of the methods provided herein, be selected for additional monitoring or interventional treatment provided to alleviate or prevent the development or worsening of such disorders. Such individuals might, as a result of the methods provided herein, be selected for additional monitoring or interventional treatment provided to enhance or assist the development of enhanced cognitive skills.

In one embodiment, the method includes the detection in a sample of cells from an individual a level of amplification (described in detail below) of the DUF1220 domain (i.e., the domain encoding DUF1220 protein). The copy number of DUF1220 domain copies in cells according to the invention can be measured, for example by array comparative genomic hybridization (array CGH), in nuclei, and the protein expression can be measured, for example in immunohistochemistry assays, Western blot analysis, in cell nuclei, cytoplasm and/or membranes. The DUF1220 domain copy number may also be measured by PCR techniques, including, especially ddPCR (Hindson et al (2011). Anal Chem 83:8604-8610). PCR, as well as other detection methods (e.g., FISH, immunohistochemistry), can be performed in cells found in biological samples such as sputum, bronchial lavage, ascites, spinal fluid, brain biopsy, blood (e.g. white blood cells) or other biological tissues or fluids. The markers can be measured in cell samples that are fresh, frozen, fixed or otherwise preserved.

According to the present invention, a probe (oligonucleotide probe) is a nucleic acid molecule which typically ranges in size from about 20-100 nucleotides to several hundred nucleotides to several thousand nucleotides in length. Therefore, a probe can be any suitable length for use in an assay described herein, including any length in the range of 20 to several thousand nucleotides, in whole number increments, specifically including 60-mer probe sequences. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length (e.g., 8-30 nucleotides) that are used in polymerase chain reactions. PCR primers and hybridization probes can readily be developed and produced by those of skill in the art, using sequence information from the target sequence.

DUF1220 sequences exist in two distinct genomic environments: as a single (ancestral) domain in PDE4DIP (myomegalin) and as multiple tandem copies in the NBPF multigene family. Only the second form has undergone an evolutionarily rapid copy-number expansion. Excluding the ancestral DUF1220 domain, the remaining DUF1220-domain-encoding DNA sequences can be divided into six subgroups, or clades, designated HLS1, HLS2, HLS3, CON1, CON2, and CON3. The copy number of DUF1220 sequences that belong to HLS clades has increased markedly in the human lineage, whereas the copy number of DUF1220 sequences belonging to CON clades is more similar across primates and nonprimate mammals. (O'Bleness, et al (2012). Evolutionary History and Genome Organization of DUF1220 Protein Domains. Genes, Genomes, Genetics doi: 10.1534/g3.112.003061 G3 (September 2012) 2(9):977-986).

In a method of the disclosure, the level of DUF1220 domain amplification (the level of the DUF1220 domain) in the cell sample is compared to a control level of DUF1220 domain selected from: (i) a control level that has been correlated with abnormally small brain size; and (ii) a control level that has been correlated with abnormally large brain size. An individual is selected as being predicted to develop microcephaly, if the level of DUF1220 domain in the individual's cells is statistically similar to or less than the control level of DUF1220 domain that has been correlated with microcephaly, or if the level of DUF1220 domain in the individual's cells is statistically less than the level of DUF1220 domain that has been correlated with normal brain size. An individual is selected as being predicted to develop macrocephaly if the level of DUF1220 domain in the individual's cells is statistically greater than or equal to the control level of DUF1220 domain that has been correlated with macrocephaly, or if the level of DUF1220 domain in the individual's cells is statistically greater than the level of DUF1220 domain that has been correlated with normal brain size.

In a related method, the level of DUF1220 domain amplification in the cell sample is compared to a control level of DUF1220 domain selected from: (i) a control level that has been correlated with a diagnosis of microcephaly; and (ii) a control level that has been correlated with a diagnosis of macrocephaly. An individual is selected as being predicted to develop microcephaly, if the level of DUF1220 domain in the individual's cells is statistically similar to or less than the control level of DUF1220 domain that has been correlated with a diagnosis of microcephaly, or if the level of DUF1220 domain in the individual's cells is statistically less than the level of DUF1220 domain that has been correlated with normal brain size. An individual is selected as being predicted to develop macrocephaly if the level of DUF1220 domain in the individual's cells is statistically greater than or equal to the control level of DUF1220 domain that has been correlated with a diagnosis of macrocephaly, or if the level of DUF1220 domain in the individual's cells is statistically greater than the level of DUF1220 domain that has been correlated with normal brain size.

In a related method, the copy number of the CON2 sub-type of DUF1220 domain (i.e., the level of DUF1220 (CON2) domain amplification in the cell sample) is compared to a control level of DUF1220 domain selected from: (i) a control level that has been correlated with an IQ below 100; and (ii) a control level that has been correlated with an IQ above 140. An individual is selected as being predicted to have a low IQ, if the level of DUF1220 domain in the individual's cells is statistically similar to or less than the control level of DUF1220 domain that has been correlated with an IQ below 100, or if the level of DUF1220 domain in the individual's cells is statistically less than the level of DUF1220 domain that has been correlated with an IQ between 100 and 140. An individual is selected as being predicted to have a high IQ if the level of DUF1220 domain in the individual's cells is statistically greater than or equal to the control level of DUF1220 domain that has been correlated with an IQ above 140, or if the level of DUF1220 domain in the individual's cells is statistically greater than the level of DUF1220 domain that has been correlated with an IQ between 100 and 140. For the specific detection of the CON2 sub-type DUF1220 domain, individuals having greater than 33 copies of the CON2 sub-type DUF1220 domain are predicted to have an IQ greater than 140. Conversely, individuals having less 26 copies of the CON2 sub-type DUF1220 domain are predicted to have an IQ less than 100.

More specifically, according to the present invention, a “control level” is a control level of domain copy number, which can include a level that is correlated with any one of normal brain function, normal brain size, normal cognitive function, abnormally small brain size, abnormally large brain size, low cognitive function, high cognitive function, a diagnosis of microcephaly, a diagnosis of macrocephaly, an IQ score below 100, an IQ score above 140, and an IQ score between 100 and 140.

It will be appreciated by those of skill in the art that a control level need not be established for each assay as the assay is performed but rather, a baseline or control can be established by referring to a form of stored information regarding a previously determined control level for small or large brain size, low or high cognitive function, or low or high IQ. Such a form of stored information can include, for example, but is not limited to, a reference chart, listing or electronic file of population or individual data regarding population statistics, or any other source of data regarding control DUF1220 levels that is useful for the individual to be evaluated. For example, one can use the guidelines established above and further described in the Examples for establishing DUF1220 domain levels, which have already been correlated with responsiveness to an DUF1220 inhibitor, to rate a given individual sample.

In one embodiment, the method includes a step of detecting the expression of DUF1220 protein. Protein expression can be detected in suitable tissues, such as tissue and cell material obtained by biopsy. For example, the individual biopsy sample, which can be immobilized, can be contacted with an antibody, an antibody fragment, or an aptamer, that selectively binds to the protein to be detected, and determining whether the antibody, fragment thereof or aptamer has bound to the protein. Protein expression can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

Another embodiment of the invention includes an assay kit for performing any of the methods of the present invention. The assay kit can include any one or more of the following components: (a) a means for detecting in a sample of cells a level of DUF1220 domain; (b) a means for detecting in a sample of cells the expression of DUF1220 protein; and/or (c) a means for detecting in a sample of cells at least one (but can include more than one) mutations in the DUF1220 domain. The assay kit preferably also includes one or more controls. The controls may include: (i) a control sample for detecting low DUF1220 domain levels in an individual; (ii) a control sample for detecting high DUF1220 domain levels in an individual; (iii) information containing a predetermined control level of the DUF1220 domain. The assay kit may also include a control gene with a known copy number that can serve as an internal standard to which DUF1220 copy number measurements can be compared. The assay kit may also include means for measuring the copy number of a known gene that can serve as an internal standard to which DUF1220 copy number measurements can be compared. In a specific embodiment, the control gene is the Ribonuclease P protein subunit p30 (RPP30) gene.

In one embodiment, a means for detecting DUF1220 domain level can generally be any type of reagent that can be used in a method of the present invention. Such a means for detecting include, but are not limited to: a probe or primer(s) that hybridizes under stringent hybridization conditions to the DUF1220 domain or a portion of chromosome 1 (the chromosome on which DUF1220 is located) or probe/primer sequences that can be used for PCR-based methods that measure DUF1220 copy number at the DNA level (for example ddPCR techniques). The nucleic acid sequence for the DUF1220 domain is known in the art and can be used to produce such reagents for detection. Additional reagents useful for performing an assay using such means for detection can also be included, such as reagents for performing in situ hybridization, reagents for detecting fluorescent markers, reagents for performing polymerase chain reaction, and the like.

The means for detecting in the assay kit of the present invention can be conjugated to a detectable tag or detectable label. Such a tag can be any suitable tag which allows for detection of the reagents used to detect the gene or protein of interest and includes, but is not limited to, any composition or label detectable by spectroscopic, photochemical, electrical, optical or chemical means. Useful labels in the present invention include: biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting in the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The kit can also include a set of written instructions for using the kit and interpreting the results.

The kit may also include a means for detecting a control marker that is characteristic of the cell type being sampled, that can generally be any type of reagent that can be used in a method of detecting the presence of a known marker (at the nucleic acid or protein level) in a sample. Means for detecting a control marker include, but are not limited to: a probe that hybridizes under stringent hybridization conditions to a nucleic acid molecule encoding a protein marker; PCR primers which amplify such nucleic acid molecule; an aptamer that specifically binds to a conformationally distinct site on the target molecule; and/or an antibody, antigen binding fragment thereof, or antigen binding peptide that selectively binds to the control marker in the sample. Nucleic acid and amino acid sequences for many cell markers are known in the art and can be used to produce reagents for detection.

The assay kits and methods of the present invention can be used not only to identify individuals that are predicted to develop small or large brain size, or high or low cognitive function, but also to identify treatments that can improve cognitive function or ameliorate a cognitive dysfunction associated with low DUFF1220 domain levels.

The present disclosure is also concerned with methods for enhancing congnitive function and cognition in a mammal by increasing DUF1220 expression in the mammal, or administering the peptide expression products of DUF1220, or fragments thereof, or mimetics thereof, to the mammal.

The present invention is also concerned with methods for preventing or treating various diseases, for example, stroke, neurodegenerative diseases, anxiety, depression, memory loss, and cognitive disorders, described in this invention using modulators of DUF1220 expression or the peptide expression products of DUF1220, or by elevating the copy number of the DUF1220 domain in an individual.

This invention also relates to a method of modulating DUF1220 expression and subsequent neurogenesis in brain cells and brain tissues, memory formation and improvement in cognitive abilities and improvement in IQ. Thus, this invention provides a method of treating or preventing stroke, anxiety, depression, memory loss and cognitive disorders using DUF1220 inhibitors, antagonists, stimulants, agonists, and/or their derivatives. The invention may also be important to the diagnosis and treatment of schizophrenia and autism, which have been associated with smaller and larger brain size or brain growth parameters, respectively. Copy number variations in the genomic region where most DUF1220 copies map have been linked to schizophrenia (deletions) and autism (duplications).

More embodiments concern methods of screening for therapeutic agents useful in the treatment of a neurological diseases or the enhancement of cognition in a human comprising contacting a test compound with a polypeptide expression product of DUF1220; and detecting binding of said test compound to said polypeptide. Additional embodiments provide methods of screening for therapeutic agents useful in the treatment of a neurological disease or enhancing cognition in a mammal comprising (a) determining the activity of any one of the polypeptides described above, at a first concentration of a test compound or in the absence of said test compound, (b) determining the activity of said polypeptide at a second concentration of said test compound, and comparing the activity of said polypeptide under conditions (a) and (b) to the activity of the polypeptide in the presence of a known regulator. In some embodiments, the activity is current.

Additional aspects concern a method for the preparation of a pharmaceutical composition useful for the treatment of a neurological disease or enhancing cognition in a mammal comprising identifying a regulator of DUF1220 expression in the mammal, or activity of a DUF1220 peptide expression product, determining whether said regulator ameliorates the symptoms of said neurological disease or enhancing cognition in a mammal, and combining said regulator with an acceptable pharmaceutical carrier. In some embodiments, the regulator is a small molecule, an RNA molecule, an antisense oligonucleotide, a polypeptide, an antibody, or a ribozyme.

In another embodiment, the invention provides assays for screening candidate or test compounds that bind to or modulate the expression of DUF1220 or the functional activity of a peptide expression product of DUF1220. Such assays can employ full-length DUF1220, a biologically active fragment of DUF1220, or a fusion protein that includes all or a portion of DUF1220.

This Summary of the Invention is neither intended nor should it be construed as being representative of the full extent and scope of the present invention. Additional aspects of the present invention will become apparent from the following description and experimental findings.

### DESCRIPTION OF ILLUSTRATIVE EMBODIMENTS

The present disclosure is drawn to methods of assessing DUF1220 domain copy number in an individual that can be used to assess the cognitive function or neurodevelopmental status or prognosis of an individual.

Diagnostics: One embodiment of the invention relates to a method for predicting the likelihood that an individual will have a neurodevelopmental disorder, e.g., microcephaly or macrocephaly, or for aiding in the diagnosis of a neurodevelopmental disorder, or a greater likelihood of having symptomology associated with a neurodevelopmental disorder, comprising the steps of obtaining a DNA sample from an individual to be assessed and determining the DUF1220 copy number present in the individual. The rationale behind the diagnostic assay is based on the link between DUF1220 copy number and brain size: in general the more copies of DUF1220 encoded in the genome, the larger the brain size. The more extreme (higher or lower) the DUF1220 copy number, the greater the likelihood of having a brain-size-related abnormality, such as microcephaly or macrocephaly. The presence of a DUF1220 copy number at or above the 90^{th} percentile is at an increased risk of developing an abnormally large brain size, e.g. macrocephaly. Conversely, the presence of a DUF1220 copy number at or below the 10^{th} percentile is at an increased risk of developing an abnormally small brain size, e.g. microcephaly.

In a preferred embodiment, the neurodevelopmental disorder is microcephaly or macrocephaly. In a particular embodiment, the individual is an individual at risk for development of microcephaly. In another embodiment the individual exhibits clinical symptomatology associated with microcephaly. In one embodiment, the individual has been clinically diagnosed as having microcephaly. In another particular embodiment the test could be applied prenatally as a means of detecting increased risk for brain size abnormalities.

The genetic material within the individual's biological sample to be assessed can be obtained from any nucleated cell from the individual, but may also include free DNA, e.g. in blood, amniotic fluid or other human fluid. For assay of genomic DNA, virtually any biological sample (other than pure red blood cells) is suitable. For example, convenient tissue samples include whole blood, semen, saliva, tears, urine, fecal material, sweat, skin and hair. Additional samples may include fetal DNA obtained from various sources, including fetal cells, amniotic fluid and maternal blood. For assay of cDNA or mRNA, the tissue sample must be obtained from an organ in which the target nucleic acid is expressed. Neural crest-derived cells include, for example, melanocytes and keratinocytes.

Many of the methods described herein require amplification of DNA from target samples. This can be accomplished by e.g., PCR. See generally: PCR Technology: Principles and Applications for DNA Amplification ed. H. A. Erlich, Freeman Press, NY, N.Y., 1992; and specifically, Hindson B. J, Ness K D, Masquelier D A, et. al. (2011). High-throughput digital PCR system for absolute quantitation of DNA copy number. Anal. Chem. 83:8604-8610. Other suitable amplification methods include droplet digital PCR (ddPCR), the ligase chain reaction (LCR), transcription amplification, self-sustained sequence replication, and nucleic acid-based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

In conducting mutation analysis, nucleotides of interest can be identified by a variety of methods, such as Southern analysis of genomic DNA; direct mutation analysis by restriction enzyme digestion; Northern analysis of RNA; denaturing high pressure liquid chromatography (DHPLC); gene isolation and sequencing; hybridization of an allele-specific oligonucleotide with amplified gene products; single base extension (SBE); or analysis of the DUF1220 protein. Additional methods for measuring domain copy number include ddPCR (digital drop PCR), array comparative genomic hybridization (array CGH) and the use of DNA sequence read-depth strategies.

In specific embodiments, the present invention provides methods for determining whether a subject has microcephaly. In another aspect, the invention provides methods for diagnosing microcephaly in an individual. In another aspect, the invention provides methods for determining the likelihood of developing microcephaly in an individual. In another aspect, the invention provides methods for determining the likelihood that an individual will have increased symptomology associated with microcephaly. These methods comprise obtaining a biological sample from an individual suspected of having microcephaly, or at risk for developing microcephaly, detecting the DUF1220 domain copy number or expression level or activity of one or more protein expression products of the DUF1220 domain in the sample, and comparing the result to the expression level or activity of the marker(s) in a sample obtained from a non-microcephalic subject, or to a reference range or value established for non-microcephalic individuals. Similar tests and reference values may be used to determine the presence or likelihood of developing macrocephaly in an individual or for assessing the cognitive function of an individual.

As used herein, the term “biological sample” includes a sample from any body fluid or tissue (e.g., serum, plasma, blood, cerebrospinal fluid, urine, brain tissue). Typically, the standard biomarker level or reference range is obtained by measuring the same marker or markers in a set of normal controls. Measurement of the standard biomarker level or reference range need not be made contemporaneously; it may be a historical measurement. Preferably the normal control is matched to the individual with respect to some attribute(s) (e.g., age). Depending upon the difference between the measured and standard level or reference range, the individual can be diagnosed as having microcephaly or as not having microcephaly. Similarly, as having macrocephaly not having macrocephaly, or high cognitive function or low cognitive function. In some embodiments, microcephaly is diagnosed in the individual if the expression level of the biomarker or biomarkers in the individual sample is statistically more similar to the expression level of the biomarker or biomarkers that has been associated with microcephaly than the expression level of the biomarker or biomarkers that has been associated with the normal controls.

The methods of the present invention may be used to make the diagnosis of microcephaly or macrocephaly, independently from other information such as the individual's symptoms or the results of other clinical or paraclinical tests. However, the methods of the present invention may be used in conjunction with such other data points. Because a diagnosis is rarely based exclusively on the results of a single test, the method may be used to determine whether a subject is more likely than not to have microcephaly/macrocephaly, or is more likely to have microcephaly/macrocephaly than to have another disease, based on the difference between the measured and standard level or reference range of the DUF1220 domain copy number or protein products. Thus, for example, an individual with a putative diagnosis of microcephaly (e.g., suspected to be suffering from microcephaly) may be diagnosed as being “more likely” or “less likely” to have microcephaly in light of the information provided by a method of the present disclosure. If a plurality of biomarkers are measured, at least one and up to all of the measured biomarkers must differ, in the appropriate direction, for the subject to be diagnosed as having (or being more likely to have) microcephaly. In some embodiments, such difference is statistically significant.

As will be apparent to those of ordinary skill in the art, the above description is not limited to making an initial diagnosis of microcephaly/macrocephaly, but also is applicable to confirming a provisional diagnosis of microcephaly/macrocephaly or “ruling out” such a diagnosis. Furthermore, an increased or decreased level or activity of the marker(s) in a sample obtained from a subject suspected of having microcephaly, or at risk for developing microcephaly, is indicative that the subject has or is at risk for developing microcephaly.

The invention also provides a method for determining an individual's risk of developing microcephaly, the method comprising obtaining a biological sample from a subject, detecting the level or activity of a marker in the sample, and comparing the result to the level or activity of the marker in a sample obtained from a normal or non-microcephalic subject, or to a reference range or value wherein an increase or decrease of the marker is correlated with the risk of developing microcephaly.

The assay system can also include a means for detecting a control marker that is characteristic of the cell type being sampled and can generally be any type of reagent that can be used in a method of detecting the presence of a known marker (at the nucleic acid or protein level) in a sample, such as by a method for detecting the presence of the DUF1220 domain biomarker. Specifically, the means is characterized in that it identifies a specific marker of the cell type being analyzed that positively identifies the cell type.

Another aspect of the disclosure provides methods that can be used to identify individuals at risk for developing microcephaly, and/or for predicting the prognosis of an individual having microcephaly. This aspect of the disclosure relates to the discovery that the copy number of the DUF1220 domain is negatively associated with an increased risk and incidence of microcephaly. Therefore, one embodiment provides a method of diagnosing or assessing the risk of developing microcephaly in an individual including obtaining a biological sample from an individual, analyzing the sample for the copy number of the DUF1220 domain. Low copy number and/or protein expression/activity of DUF1220 indicates that the individual is at risk for developing microcephaly or is suffering from microcephaly and is likely to benefit from the administration of a drug or other treatment used to treat or support microcephalic patients. Alternatively, high expression and/or protein function of the DUF1220 domain indicates that the individual is at low risk for developing microcephaly or is unlikely to be suffering from microcephaly and is unlikely to benefit from the administration of a therapy used to treat or support a microcephalic patient. According to the present invention, the individual being tested can be a human or non-human primate. The individual may or may not be suspected of having microcephaly. In one embodiment, the individual being tested has not been diagnosed as having microcephaly. Therefore, one embodiment is methods for diagnosing microcephaly or macrocephaly in an individual or predicting the likelihood that an individual will develop microcephaly or macrocephaly or will have increased symptomology associated with microcephaly or macrocephaly comprising:
a) obtaining a biological sample from an individual;
b) detecting in the biological sample the presence of at least one marker selected from:
   i) the copy number of the DUF1220 domain; and,
   ii) the expression or activity level of a DUF1220 protein expression product;
   wherein the level of the at least one marker is indicative of microcephaly or macrocephaly in the individual and a likelihood that the individual will have increased symptomology associated with microcephaly or macrocephaly.

In this method, the biological sample may be a sample of biological fluid or a tissue sample selected from the group consisting of blood, tears, urine, saliva, skin, muscle and lymph tissue. In this method, the step of detecting may include determining the protein activity of the DUF1220 protein expression products in the sample. In this method, the step of detecting may include performing at least one technique selected from the group consisting of Sanger dideoxy sequencing, pyrosequencing, other types of DNA sequencing, RNA sequencing, single-strand conformation polymorphism, heteroduplex analysis, DNA microarray technology, denaturing gradient gel electrophoresis, allele-specific PCR, Scorpion Amplification Refractory Mutation System (SARMS) technology, SNaPshot analysis of PCR products, droplet digital PCR (ddPCR), and mass spectrometry.

Another aspect is a method of identifying compounds that increase the cognitive function in a mammal by modulating the expression or activity of the DUF1220 protein in the mammal. In some instances, the cognitive function in the mammal will be increased by enhancing the expression or activity of the DUF1220 domain protein. Determining the ability of the test compound to bind to a membrane-bound form of DUF1220 can be accomplished, for example, by coupling the test compound with a radioisotope or enzymatic label such that binding of the test compound to the DUF1220-expressing cell can be measured by detecting the labeled compound in a complex. In a competitive binding format, the assay comprises contacting a DUF1220 expressing cell with a known compound that carries a detectable label and that binds to DUF1220 to form an assay mixture, contacting the assay mixture with a test compound, and determining the ability of the test compound to interact with the DUF1220 expressing cell, wherein determining the ability of the test compound to interact with the DUF1220 expressing cell comprises determining the ability of the test compound to preferentially bind DUF1220 in the cell as compared to the known compound.

In another embodiment, the assay is a cell-based assay comprising contacting a cell expressing a DUF1220 (e.g., full-length DUF1220, a biologically active fragment of DUF1220, or a fusion protein that includes all or a portion of DUF1220) expressed in the cell with a test compound and determining the ability of the test compound to modulate (e.g., stimulate or inhibit) the functional activity of the DUF1220. Determining the ability of the test compound to modulate the functional activity of the DUF1220 can be accomplished by any method suitable for measuring the functional activity of DUF1220, e.g., any method suitable for measuring the activity. The activity can be measured in a number of ways, not all of which are suitable for any given target protein.

These methods may also include the use of cell-free assays. Such assays involve contacting a form of DUF1220 (e.g., full-length DUF1220, a biologically active fragment of DUF1220, or a fusion protein comprising all or a portion of DUF1220) with a test compound and determining the ability of the test compound to bind to DUF1220. Binding of the test compound to DUF1220 can be determined either directly or indirectly as described above. In one embodiment, the assay includes contacting DUF1220 with a known compound that carries a detectable label and that binds DUF1220 to form an assay mixture, contacting the assay mixture with a test compound, and determining the ability of the test compound to interact with DUF1220, wherein determining the ability of the test compound to interact with DUF1220 comprises determining the ability of the test compound to preferentially bind to DUF1220 as compared to the known compound.

In various embodiments of the above assay methods of the present invention, it may be desirable to immobilize DUF1220 (or a DUF1220 target molecule) to facilitate separation of complexed from uncomplexed forms of one or both of the proteins, as well as to accommodate automation of the assay. Binding of a test compound to DUF1220, or interaction of DUF1220 with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S-transferase (GST) fusion proteins can be adsorbed onto glutathione sepharose beads (Sigma Chemical; St. Louis, Mo.) or glutathione derivatized microtiter plates, which are then combined with the test compound or the test compound and either the non-adsorbed target protein or DUF1220, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of DUF1220 can be determined using standard techniques.

Other techniques for immobilizing proteins on matrices can also be used in the screening assays of the invention. For example, either DUF1220 or its target molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, IU.), and immobilized in the wells of streptavidin-coated plates (Pierce Chemical). Alternatively, antibodies reactive with DUF1220 or target molecules but that do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with DUF1220 or target molecule, as well as enzyme-linked assays that rely on detecting an enzymatic activity associated with DUF1220 or target molecule.

The screening assays can also involve monitoring the expression of DUF1220. For example, regulators of expression of DUF1220 can be identified in a method in which a cell is contacted with a candidate compound and the expression of DUF1220 protein or mRNA in the cell is determined. The level of expression of DUF1220 protein or mRNA in the presence of the candidate compound is compared to the level of expression of DUF1220 protein or mRNA in the absence of the candidate compound. The candidate compound can then be identified as a regulator of expression of DUF1220 based on this comparison. For example, when expression of DUF1220 protein or mRNA protein is greater (statistically significantly greater) in the presence of the candidate compound than in its absence, the candidate compound is identified as a stimulator of DUF1220 protein or mRNA expression. Alternatively, when expression of DUF1220 protein or mRNA is less (statistically significantly less) in the presence of the candidate compound than in its absence, the candidate compound is identified as an inhibitor of DUF1220 protein or mRNA expression. The level of DUF1220 protein or mRNA expression in the cells can be determined by methods well known in the art.

Gene Expression: In another embodiment, test compounds that increase or decrease DUF1220 domain expression are identified. As used herein, the term “correlates with expression of a polynucleotide” indicates that the detection of the presence of nucleic acids, the same or related to a nucleic acid sequence encoding DUF1220, by northern analysis or real-time PCR is indicative of the presence of nucleic acids encoding DUF1220 in a sample, and thereby correlates with expression of the transcript from the polynucleotide encoding DUF1220. The term “microarray”, as used herein, refers to an array of distinct polynucleotides or oligonucleotides arrayed on a substrate, such as paper, nylon or any other type of membrane, filter, chip, glass slide, or any other suitable solid support. A DUF1220 polynucleotide is contacted with a test compound, and the expression of an RNA or polypeptide product of DUF1220 polynucleotide is determined. The level of expression of appropriate mRNA or polypeptide in the presence of the test compound is compared to the level of expression of mRNA or polypeptide in the absence of the test compound. The test compound can then be identified as a regulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

Such screening can be carried out either in a cell-free assay system or in an intact cell. Any cell that expresses DUF1220 polynucleotide can be used in a cell-based assay system. The DUF1220 polynucleotide can be naturally occurring in the cell or can be introduced into the cell. Either a primary culture or an established cell line can be used.

Methods of Use: The present invention provides for both prophylactic and therapeutic methods for enhancing cognition in a mammal or treating neurological diseases/neurological disorders, e.g., schizophrenia, autism, autism spectrum disorder, microcephaly, or macrocephaly.

The modulatory methods involve contacting a cell with an agent that modulates one or more of the activities of DUF1220. An agent that modulates activity can be an agent as described herein, such as a nucleic acid or a protein (including the DUF1220 domain protein), a naturally-occurring cognate ligand of the polypeptide, a peptide, a peptidomimetic, or any small molecule. In one embodiment, the agent stimulates one or more of the biological activities of DUF1220. In another embodiment, the agent inhibits one or more of the biological activities of DUF1220. As such, the present invention provides methods of treating an individual afflicted with a disease or disorder characterized by unwanted expression or activity of DUF1220 or a protein expression product of DUF1220. In one embodiment, the method involves administering an agent like any agent identified or being identifiable by a screening assay as described herein, or combination of such agents that may upregulate or downregulate the expression or activity of DUF1220 or of any protein expression product of DUF1220. In another embodiment, the method involves administering a regulator of DUF1220 as therapy to compensate for increased or undesirably high expression or activity, or, alternatively, reduced or undesirably low expression or activity of DUF1220 or a protein expression product of DUF1220.

Stimulation of activity or expression of DUF1220 is desirable in situations in which activity or expression is abnormally low, or lower than desirable, and in which increased activity is likely to have a beneficial effect, e.g. enhanced cognition in the mammal. Conversely, inhibition of activity or expression of DUF1220 is desirable in situations in which activity or expression of DUF1220 is abnormally high and in which decreasing its activity is likely to have a beneficial effect.

Another aspect is a method of modulating expression and/or activity of the DUF1220 protein in a mammal to increase cognitive function in the mammal, to improve motor function, to improve memory and learning abilities, to reduce depression and anxiety, and/or to treat, ameliorate, or prevent a neurodevelopmental disorder, and/or to treat, ameliorate, or prevent a neurological disorder such as autism, schizophrenia. These embodiments may include administering to the mammal a therapeutically effective amount of a DUF1220 protein, agonist, mimic, expression enhancer, or the like. Preferably, the mammal in these embodiments is a human. Preferably, the agent administered to increase the expression and/or activity of the DUF1220 domain enhances memory and/or learning ability and/or cognition in the mammal. A specific embodiment is the use of DUF1220 protein in the manufacture of a medicament for the treatment of microcephaly or the enhancement of congnitive function in a human. A related embodiment is the use of a pharmaceutical composition comprising DUF1220 protein in the preparation of a medicament for the treatment of microcephaly or the enhancement of congnitive function in a human. Another specific embodiment is a pharmaceutical composition comprising DUF1220 protein for use in the treatment of microcephaly or the enhancement of congnitive function in a human. Another embodiment is a method of treating microcephaly or the enhancing congnitive function in an individual comprising administering an effective amount of DUF1220 protein to an individual in need thereof. Another embodiment is a method of treating an individual suffering from, or at risk of developing microcephaly or low cognitive function comprising administering an effective amount of DUF1220 protein to the individual in need thereof.

Pharmaceutical Compositions: This invention further pertains to isolated proteins or novel agents identified by the above-described screening assays and uses thereof for the therapeutic uses described above. The active agents (also referred to herein as “active compounds”) of the invention can be incorporated into pharmaceutical compositions suitable for administration. Such compositions typically comprise the active agents and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes pharmaceutical compositions comprising a regulator of DUF1220 expression or activity (and/or a regulator of the activity or expression of a protein in the DUF1220 signaling pathway) as well as methods for preparing such compositions by combining one or more such regulators and a pharmaceutically acceptable carrier.

An antagonist of DUF1220 may be produced using methods that are known in the art. In particular, purified DUF1220, or polypeptides corresponding to all or part of the DUF1220 domain, may be used to produce antibodies or to screen libraries of pharmaceutical agents to identify those that specifically bind DUF1220. Antibodies to DUF1220 may also be generated using methods that are well known in the art. Such antibodies may include, but are not limited to, polyclonal, monoclonal, chimeric, single chain antibodies, Fab fragments, and fragments produced by a Fab expression library.

In another embodiment, the polynucleotides encoding DUF1220, or any fragment or complement thereof, may be used for therapeutic purposes. In one aspect, the complement of the polynucleotide encoding DUF1220 may be used in situations in which it would be desirable to block the transcription of the DUF1220 mRNA. In particular, cells may be transformed with sequences complementary to polynucleotides encoding DUF1220. Thus, complementary molecules or fragments may be used to modulate DUF1220 activity, or to achieve regulation of gene function. Such technology is now well known in the art, and sense or antisense oligonucleotides or larger fragments can be designed from various locations along the coding or control regions of sequences encoding DUF1220.

Any of the therapeutic methods described above may be applied to any subject in need of such therapy, including, preferably, humans.

A related embodiment is the administration of a pharmaceutical composition containing DUF1220 in conjunction with a pharmaceutically acceptable carrier, for any of the therapeutic effects discussed above. Such pharmaceutical compositions may constitute DUF1220, antibodies to DUF1220, and mimetics, agonists, antagonists, or inhibitors of DUF1220. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

The Examples, which follow, are illustrative of specific embodiments of the invention, and various uses thereof. They are set forth for explanatory purposes only, and are not to be taken as limiting the invention.

### EXAMPLES

### Example 1

### DUF1220—Domain Copy Number and Brain-Size Pathology and Evolution

This example demonstrates the use of specialized bioinformatics tools developed for scoring highly duplicated DUF1220 sequences to implement targeted 1q21 array comparative genomic hybridization on individuals (n=42) with 1q21-associated microcephaly and macrocephaly.

DUF1220 Copy Number versus Brain Graphs: DUF1220 association with copy number, brain weight, and cortical neuron counts were graphed with Excel. The relationships were evaluated by ordinary least-squares (simple linear) regression with R version 2.10.1. Brain weights were taken from the literature.

Genomic DNA Samples: The Medical Genetics Laboratories (Cytogenetic and Microarray Laboratories) at Baylor College of Medicine provided DNA isolated from the blood of individuals affected with microcephaly or macrocephaly. The samples provided included 28 individuals with previously reported 1q21 deletions or duplications and microcephaly or macrocephaly and were ascertained from a larger survey of more than 16,000 individuals. From the same laboratory, an additional 14 samples, which were not previously assayed on low-resolution arrays, were included in this study. Collaborating labs from NIMH provided DNA samples (extracted from immortalized cell lines) from normal individuals at the extremes of high and low brain size. The presence of extreme brain size was determined on the basis of residual volumes of total brain, total gray matter, and total white matter after age and sex were accounted for in a large (n>300) brain structural magnetic resonance image (sMRI) database. Brain sMRI scans were all T-1 weighted images with contiguous 1.5 mm axial slices and 2.0 mm coronal slices and were obtained on the same 1.5-T General Electric Signa scanner with a three-dimensional spoiled gradient-recalled echo sequence. The volumetric indices were derived for each scan with the use of a well-validated, and fully automated image-processing pipeline. All procedures followed were in accordance with the ethical standards of the responsible committee on human experimentation (institutional and national), and proper informed consent was obtained. Design of Custom High-Density 1q21.1-1q21.2 DNA Microarrays: In order to detect CNVs in DUF1220-domain-encoding sequences in the 1q21.1-1q21.2 region of the genome among individuals, we designed high-density custom human microarrays by using Agilent Technologies 8×60K platform. The array was enriched for both unique and nonunique 60-mer probe sequences that mapped to Chr 1: 141-150 Mb. The arrays were designed prior to the release of the 2009 human genome assembly (hg19) and were therefore generated with the 2006 human genome assembly (hg18). The nonunique probes from chromosome 1, including DUF1220-domain-encoding sequences, were specific to 1q21 gene regions. On average, the probe coverage on chromosome 1 included 8-9 probes per 1 kb. A total of 43,010 probes were located on chromosome 1. The remaining 15,500 probes were randomly chosen and mapped to unique regions from the rest of the genome (hg18). aCGH Assays Oxford Gene Technology (OGT) performed the array comparative genomic hybridization (aCGH) as an Agilent certified service provider. OGT utilized the following protocols for preparation of the test and reference samples: Agilent Oligonucleotide Array-Based CGH for Genomic DNA Analysis, Enzymatic Labeling for Blood, Cells, or Tissues (with a High Throughput option) version 6.3, October 2010 (G4410-90010); and Agilent Oligonucleotide Array-Based CGH for Genomic DNA Analysis, Enzymatic Labeling for Blood, Cells, or Tissues (with a High Throughput option) version 6.2.1, February 2010 (G4410-90010). The Agilent Genomic DNA Enzymatic Labeling Kit (Agilent p/n 5190-0449) was used for labeling the samples. Protocols were adhered to with the restriction-digest option and with the 96-well clean-up option (AutoScreen-96A Well plates GE Healthcare p/n 25-9005-98). All slides were washed twice before scanning. Slides were scanned on an Agilent C scanner under ozone-controlled conditions. Data were extracted from the TIFF images with Agilent Feature Extraction software (version 10.7.1). All sample- and slide-processing steps and QC metrics were recorded in OGT's Laboratory Information Management System. So that all test samples could be compared to one another, the same single unaffected male DNA sample was used as a reference sample for all experiments.

Analysis of aCGH Data: The custom 1q21 microarray was composed of probes corresponding to both unique and nonunique sequences, and approximately 75% of the probes were located on chromosome 1. The nonunique probes have sequences that are found at multiple loci in the human genome, and the majority of them are located within the highly duplicated DUF1220-domain-encoding regions. The original design of the microarray probe sequences was based on the hg18 2006 assembly, which was converted to the hg19 build as follows. All 57,897 genomic probe sequences on the microarray were aligned to the hg19 2009 assembly with BLAT. If a BLAT search resulted in a match of at least 59 of 60 nucleotides of each probe sequence, that probe sequence was retained and mapped according to the 2009 assembly and was used in the aCGH analysis. This remapping of the original 57,897 probes resulted in 267,841 genomic probe loci in the data set so that the nonunique probes were remapped to all loci determined by the BLAT alignment results. The log 2 ratios for the nonunique array probes were assigned to each newly mapped nonunique probe locus. In summary, a specific probe that was originally assigned to one genomic locus was remapped to multiple loci. The log 2 ratios for probes that mapped to the same location were averaged. Specific steps of the aCGH analysis are as follows. For each array experiment, the TIFF image of the array was imported into Agilent Feature Extraction Software version 10.7.3.1 for image analysis, which included dye-bias normalization and data extraction. The dye-bias normalization excluded all probes on chromosomes 1, X, and Y. The chromosome 1 probes were omitted for normalization methods because the majority of these probes were not unique on the basis of the array design. Additionally, probes on chromosomes X and Y were omitted for alleviating any bias created from test and reference samples that were not matched for sex. So that data could be compared between experiments, an acrossarray normalization was carried out as follows. After the dyenormalized log 10 ratio for each probe was converted to a linear ratio, all probes corresponding to a given target sequence or region of interest (e.g., DUF1220 clade, non-NBPF gene, etc.) were averaged for an average linear ratio for that gene or region. Next, all single-locus probes (i.e., probes corresponding to single-copy genome sequences excluding chromosomes 1, X, and Y) were averaged. An adjusted score, for which the average linear ratio for a target region was divided by the average linear ratio of all single-locus probes, was then generated. Finally, the resulting normalized linear ratio was then converted into a log 2 ratio, which was then used for statistical analyses. Cytosure Interpret Software was used for viewing segmentations across chromosome 1. So that bias could be reduced from cross-hybridizing probes aligning to multiple clade types (e.g., HLS1 and CON3), in the clade analysis all nonunique probes mapping to multiple clade types (21%) were removed from the analysis. The nonunique probes that mapped to only a single clade type were retained, and the values assigned to each clade type represent an average of the signals obtained for each probe within a clade type. However, some cross-hybridization of probes to multiple genomic regions of the same clade type, such as to multiple HLS1 regions, remained. The region from each 1q21 gene's transcription start coordinate to each gene's transcription stop coordinate was divided into nonoverlapping windows of 500 bases so that the array results could be viewed for each 1q21 gene. For each sample tested, the log 2 ratio values for all probes whose coordinates mapped within each window were averaged and plotted for each gene. For viewing the DUF1220 regions on the basis of clade classification, the sequence spanning each DUF1220 repeat (clade span) located in the 1q21 region was used as a separate window and the average log 2 ratio was calculated on the basis of all probes that aligned within each window. Each DUF1220 window was first ordered by its clade classification and then by its genomic coordinate within each clade classification. qPCR Analysis Quantitative-real time PCR (qPCR), with the use of Taqman master mix on an Applied Biosystems 7300 Real-Time PCR system, was carried out on genes for each individual with optimal primer and fluorogenic probe sets that are unique to the DNA sequence of the gene of interest. Optimal primers and probes were designed with PrimerExpress (Applied Biosystem software). The amplicon sequence was used as a BLAT query against the human March 2006 (hg18) assembly for verifying that the primer and designs were sound. The functionality of each primer pair was then verified with the UCSC database for in silico PCR. Relative copy-ratio estimates were generated with the standard curve method, normalized to CFTR, cystic fibrosis transmembrane conductance regulator (ATP-binding cassette subfamily C, member 7), an ATP-binding cassette that was used as a control gene thought to represent one gene per haploid human genome. Reactions were carried out in triplicate per plate run and replicated in at least three separate plate reaction runs. In total, DNA was assayed at least nine times per individual. Copy ratios of all assays were averaged for the final ratio measure. Additionally, qPCR derived copy ratio of DUF1220 domains was compared to aCGH copy ratio on 26 individuals with known CNVs in the 1q21.1-1q21.2 region. The qPCR primer and probe sequences are as follows:

### DufQ8IX62

Forward Primer Sequence (5′-3′):
(SEQ ID NO: 1)
GCTGGAGGTAGTAGAGCCTGAAGTC
Reverse Primer Sequence (5′-3′):
(SEQ ID NO: 2)
GGAGTCAGGCTGTTCAAGACAA
Probe Sequence (5′-3′):
(SEQ ID NO: 3)
[6-FAM]TGCAGGACTCACTGGATAGATGTTATTCAACTCC[TAMRA-
6-FAM]

### Hydin

Forward Primer Sequence (5′-3′):
(SEQ ID NO: 4)
TGTGAGCAGCATGTGGACTACA
Reverse Primer Sequence (5′-3′):
(SEQ ID NO: 5)
TCAGGAGAGATGGTGAATTCTTTTG
Probe Sequence (5′-3′):
(SEQ ID NO: 6)
[6-FAM]AAGACCATCTTGGACCAAGGAAGAAATATCCTC[TAMRA-
6-FAM]

### RP11-102F23

Forward Primer Sequence (5′-3′):
(SEQ ID NO: 7)
CCTTCCAGGCCAGCTTTTG
Reverse Primer Sequence (5′-3′):
(SEQ ID NO: 8)
CGAAGCCTTTCAGATTACTCATGA
Probe Sequence (5′-3′):
(SEQ ID NO: 9)
[6-FAM]CATGCTTCCCTTCTTTCCCTCTACCCTG[TAMRA-6-FAM]

### PDE4DIP

Forward Primer Sequence (5′-3′):
(SEQ ID NO: 10)
GTCCGGGATGTTGGTATGAATT
Reverse Primer Sequence (5′-3′):
(SEQ ID NO: 11)
CCAAGCCATTTGCTCTGTTGA
Probe Sequence (5′-3′):
(SEQ ID NO: 12)
[6-FAM]TCCTCTACTCCTGGCTCAGAAACGCCC[TAMRA-Q]

### Class I-Specific

Forward Primer Sequence (5′-3′):
(SEQ ID NO: 13)
CGAAACCACTTGGCCTTCAG
Reverse Primer Sequence (5′-3′):
(SEQ ID NO: 14)
GCTTTCAGCTTTCGTAAATATTCAGTT
Probe Sequence (5′-3′):
(SEQ ID NO: 15)
[6-FAM]CCCTGCCTCGGCCAGAGGTTTC[TAMRA-6-FAM]

### Class II-Specific

Forward Primer Sequence (5′-3′):
(SEQ ID NO: 16)
TGGCTTCTTCCTGCGAATTG
Reverse Primer Sequence (5′-3′):
(SEQ ID NO: 17)
TCTGCTGGGCTACACTTCTCAA
Probe Sequence (5′-3′):
(SEQ ID NO: 18)
[6-FAM]AAGGACACCGAGGGCCACCTGG[TAMRA-6-FAM]

Statistical Analyses: To test the hypothesis that DUF1220 copy-number ratio is associated with head circumference, we tested for association between the copy number of each of the sequences of interest and frontal occipital circumference (FOC) Z scores via linear regression models. Because of a priori hypotheses regarding the biologic relevance of DUF1220 copy number to brain size, our primary inference was based on six tests that we performed by using the 42 samples from individuals with either microcephaly or macrocephaly. One test was conducted for each of the three conserved DUF1220 clades (CON1, CON2, and CON3), and one was conducted for each of the HLS clades (HLS1, HLS2, HLS3). In each case, the average of the log 2 ratio of the individual sequences was used as the measure for a clade. Secondary analyses were stratified according to whether an individual had a deletion (n=26) or a duplication (n=16) in the 1q21.1-1q21.2 region. On the basis of the results of the stratified analyses, we conducted tertiary (exploratory) analyses to test for association between each of the other genes assayed by aCGH and the FOC Z scores among the deletion subgroup. The ethnic distribution of individuals with known deletions or duplications was categorized as 26 white, 12 Hispanic, and 3 African American or other. Differences of copy ratio between ethnic groups were tested with a one-way ANOVA. We assessed population stratification in the group with a known deletion by including ethnicity as a covariate in a linear regression model of FOC Z score and CON1. There was no evidence of (1) a significant difference of copy ratio between ethnic groups (p>0.10), (2) confounding by ethnicity, or (3) an association between ethnicity and FOC Z scores. Given the lack of ethnic associations, results presented below are from unadjusted regression models. For aCGH studies of a nondisease population, a sample of 59 unrelated non-Hispanic white individuals was selected from a cohort of greater than 300 individuals for extremes in brain size. For this selection, gray-matter residual volumes were obtained from a regression controlling for sex and age. The 59 individuals selected for analysis had gray-matter residual volumes greater than 0.5 (large group, n=29) and less than −0.5 (small group, n=30). We selected extremes in phenotypes to potentially increase our power to detect differences in copy ratio between groups. We selected non-Hispanic white individuals to control for potential population stratification. This population was 69.5% male and had a mean age of 10.9 years and a standard deviation (SD) of 3.0 years. We first tested for association between each of the six DUF1220 clades and having a large or small gray-matter residual volume (on the basis of the selection criteria described above) via t tests. Lastly, in an exploratory analysis, we tested for association of large or small gray-matter volume versus aCGH-predicted copy ratio of other genes in the 1q21 region. Analyses were conducted with R version 2.10.1.

Results Microcephaly and Macrocephaly: To directly investigate the possible involvement of DUF1220 copy number in 1q21.1-1q21.2 microcephaly and macrocephaly, we designed custom 1q21 microarrays to more comprehensively cover the 4.4 Mb 1q21.1-1q21.2 interval, as well as the sequences flanking the interval. In addition, we developed new bioinformatic tools that allowed the copy number of different types of DUF1220 sequences to be independently assessed. The arrays were used for high-resolution aCGH analysis of 42 individuals with either 1g21.1-1g21.2 deletions (class I [smaller] or class II [larger]) or duplications. As originally defined by Brunetti-Pierri et al. (Nat. Genet. 40, 1466-1471.) class I deletions include a deletion in the distal 1q21.1-1q21.2 region (hg18), whereas class II deletions are larger and include the TAR syndrome (MIM 274000) region in addition to the 1q21.1-1q21.2 region. Of these individuals, 28 harbor 1q21 CNVs associated with microcephaly and macrocephaly and were identified from a previous, low-resolution genome-wide aCGH survey of>16,000 individuals.6 Also tested were an additional 14 individuals who had similar 1q21 CNVs and who were not previously described. In all cases, the same reference sample, a single unaffected male, was used, allowing all test samples to be indirectly compared to one another. For those samples (n=28) that had been previously analyzed by lower-resolution aCGH, resulting custom 1q21 array data confirmed what had been previously reported but also allowed specific measurement of DUF1220 copy number and more detailed coverage of the 1q21 region. In order to visualize DUF1220-specific aCGH signals, we plotted data for each of the 241 human DUF1220-domain encoding copies in the 1q21.1-1q21.2 region in six clade-specific groupings. Resulting data profiles for each individual tested from the disease population show that, overall, the class II deletion group lost more DUF1220 copies than did the class I deletion group for all six DUF1220 clades, whereas the duplication group gained DUF1220 sequences. qPCR independently confirmed these trends. aCGH-predicted copy-number values of DUF1220-domain-encoding sequences (included in NBPF genes) and non-NBPF sequences within the 1q21.1-1q21.2 region were next compared to head-circumference values (FOC Z scores) for each sample. The resulting data from the entire disease population (class I and class II deletion groups and the duplication group) indicate that the copy number of DUF1220 sequences (clades CON1-CON3 and HLS1-HLS3) shows a strong correlation with FOC Z scores, as shown in the following table:

Correlation between aCGH-Predicted DUF1220 Clade Copy
Number and FOC Z Scores for the Entire Disease Population

| Clade | Beta | SE | R2 | p Value |
|---|---|---|---|---|
| CON1 | 7.301 | 1.151 | 0.501 | 1.56 × 10−7 |
| CON2 | 7.125 | 1.136 | 0.496 | 1.97 × 10−7 |
| CON3 | 6.826 | 1.172 | 0.459 | 8.29 × 10−7 |
| HLS1 | 4.067 | 1.021 | 0.284 | 0.0003 |
| HLS2 | 3.703 | 0.978 | 0.264 | 0.0005 |
| HLS3 | 3.599 | 0.969 | 0.256 | 0.0006 |

Significant (p ≦ 0.05) associations are shown in bold.
Beta is the effect estimate of 1 unit increase in copy ratio from a linear regression model.
The following abbreviation is used: SE, standard error of beta.

In addition, a number of other genes within the region also exhibit a significant correlation with FOC Z scores, confirming results from the previous low-resolution aCGH analysis of these samples. To determine which sequences were driving this association, we stratified the samples by deletion (class I and class II combined) or duplication groups in the 1q21 region. Analysis of the duplication group alone demonstrated no evidence of association between head circumference and any 1q21.1-1q21.2 sequences, including those of DUF1220. However, using array data from the combined class I and class II deletion groups, we found an association between head circumference and the copy number of each of the six DUF1220 clades. The strongest association was obtained with the three evolutionarily conserved DUF1220 clades (CON1, p=0.0079; CON2, p=0.0134; and CON3, p=0.0116), whereas a significant, though more modest, association was found for the three HLS clades (HLS1, p=0.0476; HLS2, p=0.0431; and HLS3, p=0.0444). Interestingly, all NBPF (i.e., DUF1220-encoding) genes that map to 1q21 showed a significant association (p<0.044) with head circumference (on the basis of FOC Z scores) in the deletion group. Except for C1orf54, no significant association was found (p<0.05) for any of the 40 non-DUF1220-encoding genes in the critical region or adjacent to the implicated deletion interval. Although C1orf54 shows a correlation with head circumference in the deletion group, this gene is found outside the critical region for microcephaly and macrocephaly, as previously defined by Brunetti-Pierri, supra and shows no significant association with FOC Z scores across the full disease population (i.e., 1q21-associated microcephaly and macrocephaly). It also does not exhibit correlative evolutionary evidence as seen in the dramatic copy-number increase of DUF1220 sequences. For the above reasons, C1orf54 is most likely a false-positive association. Taken together, these findings implicate loss of DUF1220 copy number in 1q21-associated microcephaly. In addition, it should be noted that these results do not eliminate an increase in DUF1220 copy number as the likely cause of 1q21-associated macrocephaly in these samples, although definitive proof of its involvement will most likely require analysis of additional samples and/or finer copy-number measurements. The change in FOC-Z-score distribution across these disease samples reflects a gradual, rather than abrupt, profile, suggesting that any gene (or domain) whose dosage underlies these changes should also show a similar distribution. Indeed, unlike single- or low-copy sequences, the high copy number of DUF1220 sequences allows them to be incrementally reduced over an extremely broad copy-number range. For example, singlecopy 1q21 genes, such as BCL9, show only two discrete array profiles: one for the deletion groups and one for the duplication group. Genes, such as GPR89, with a copy in the intervals for both the class I and class II deletion groups, show only three primary levels of copy number. In contrast, DUF1220 clades, such as those from NBPF20, show a continuous copy-number profile that closely resembles the gradual distribution of FOC Z scores across these samples. Nondisease Population To investigate whether DUF1220 copy number might contribute to population variation in a brain-size-related phenotype in a nondisease population, we implemented the same custom 1q21.1-1q21.2 aCGH analysis on 59 individuals with extremes of normal variation of brain gray-matter volume. Although this phenotype is distinct from that found in the disease population, it provides an analysis of a related brain phenotype in an independently generated, nondisease cohort. After classifying individuals into large and small gray-matter groups, we tested DUF1220 sequences for mean copy-number differences between groups by using t tests. The mean CON1 and mean CON2 copy-number values were both significantly greater in the large gray-matter group than in the small gray-matter group (p=0.0246 and p=0.0334, respectively), as shown in the following table:

aCGH-Predicted Correlation of DUF1220 Clade Copy
Number versus Residual Gray Matter for the
Nondisease Population

| Clade | Beta | SE | p Value |
|---|---|---|---|
| CON1 | 0.052 | 0.023 | 0.0246 |
| CON2 | 0.042 | 0.019 | 0.0334 |
| CON3 | 0.019 | 0.020 | 0.3307 |
| HLS1 | 0.001 | 0.030 | 0.9753 |
| HLS2 | 0.017 | 0.035 | 0.6315 |
| HLS3 | 0.014 | 0.035 | 0.6881 |

Significant (p ≦ 0.05) associations are shown in bold.
Beta is the difference in mean copy ratio between high and low gray matter volume groups.

Notably, the CON1 clade also showed the strongest association with head circumference in the deletion group of the disease population. Subsequent analysis, which included 40 additional genes not related to NBPF genes but located in the 1q21.1-1q21.2 region, resulted in the ancestral DUF1220-containing gene, PDE4DIP, and four non-DUF1220-containing genes (SEC22B, NUDT17, SV2A, and SF3B4) showing a significant association between gray-matter volume and aCGH-predicted copy number (p<0.048) shown in the following table:

Correlation of Average aCGH-Based Copy Ratio of
Non-DUF1220 Genes in the 1q21.1-1q21.2
Region with Residual Gray Matter Groups for the
Nondisease Population

| Gene | Beta | SE | p Value |
|---|---|---|---|
| SRGAP2 | −0.022 | 0.018 | 0.238 |
| PDE4DIP* | 0.021 | 0.010 | 0.044 |
| SEC228 | 0.018 | 0.009 | 0.045 |
| NOTCH2NL | 0.050 | 0.032 | 0.127 |
| HFE2 | −0.017 | 0.019 | 0.369 |
| TXNIP | −0.009 | 0.011 | 0.446 |
| POLR3 | −0.014 | 0.015 | 0.351 |
| ANKRD34 | −0.051 | 0.427 | 0.233 |
| ANKRD35 | −0.030 | 0.020 | 0.131 |
| LIX1L | −0.010 | 0.008 | 0.182 |
| RBM8A | −0.021 | 0.026 | 0.424 |
| GNRHR2 | −0.021 | 0.013 | 0.108 |
| PEX11B | −0.016 | 0.018 | 0.381 |
| ITGA10 | −0.035 | 0.029 | 0.229 |
| NUDT17 | −0.037 | 0.017 | 0.034 |
| RNF115 | 0.002 | 0.009 | 0.846 |
| CD160 | −0.010 | 0.013 | 0.452 |
| PDZK1 | −0.011 | 0.008 | 0.218 |
| GPR89 | 0.006 | 0.005 | 0.298 |
| HYDIN | −0.063 | 0.127 | 0.623 |
| PRKAB2 | −0.006 | 0.005 | 0.266 |
| PDIA3P | 0.002 | 0.016 | 0.885 |
| FMO5 | −0.002 | 0.008 | 0.782 |
| CHD1L | −0.003 | 0.005 | 0.554 |
| BCL9 | −0.011 | 0.007 | 0.135 |
| ACP6 | −0.016 | 0.012 | 0.185 |
| GJA5 | −0.038 | 0.023 | 0.104 |
| GJA8 | −0.014 | 0.014 | 0.319 |
| FCGR1 | −0.005 | 0.056 | 0.936 |
| SV2A | −0.067 | 0.033 | 0.048 |
| BOLA1 | −0.054 | 0.030 | 0.072 |
| MTMR11 | −0.031 | 0.024 | 0.201 |
| OTUD7B | −0.010 | 0.007 | 0.148 |
| SF3B4 | −0.056 | 0.022 | 0.012 |
| VPS45 | 0.001 | 0.007 | 0.856 |
| PLEKHO1 | −0.021 | 0.022 | 0.329 |
| ANP32E | −0.016 | 0.012 | 0.163 |
| PRPF3 | −0.002 | 0.009 | 0.832 |
| C1orf54 | 0.001 | 0.015 | 0.927 |
| MRPS21 | −0.009 | 0.009 | 0.358 |

aCGH-predicted correlation of 1q21.1 gene copy number versus FOC Z score for nondisease populations is reported. Significant (p ≦ 0.05) associations are shown in bold.

However, three of these genes (NUDT17, SV2A, and SF3B4) show a negative correlation with gray matter and thus exhibit an inverse relationship with brain size. Additionally, SEC22B, SV2A, and SF3BF lie outside the critical 1q21.1-1q21.2 region previously linked to microcephaly and macrocephaly, and none of the four genes show an association with brain size in the disease population. Finally, unlike the close parallel that DUF1220-domain copy number shows with evolutionary changes in brain size, none of these genes exhibit such complementary evolutionary evidence (all four genes are single copy across primate lineages). These observations strongly suggest that these genes most likely represent false-positive correlations. Importantly, none of the other non-DUF1220-containing genes that map within the critical region show any correlation with head circumference in the group of healthy individuals and are probably not influencing human brain size under normal conditions. The very strong association identified for CON1 (p=1.56×10−7), CON2 (p=1.97×10−6), and CON3 (p=8.29×10−7) in the entire disease population is most likely indicative, in part, of the substantial range in FOC Z scores (>54 SDs) observed in this group. In contrast, the sample of healthy individuals, does not exhibit such wide variation, which ranges only from<53 SDs, even though it shows head-circumference extremes. For these reasons, the more modest, but still significant, association between gray-matter residual values and CON1 and CON2 (p=0.0246 and p=0.0334, respectively) could be expected given the relatively small sample size and the comparably lower range of brain volumes in a group of healthy individuals.

### Example 2

### ddPCR Protocol

This example demonstrates assessing the DUF1220 domain, CON2 subtype, copy number using Droplet digital PCR (ddPCR) (Hindson et al (2011) Anal Chem 83:8604-8610). Briefly, genomic DNA is digested with the restriction enzyme DDE1, and the product is diluted to 2 ng/ul. This product is added to a PCR mix containing primers to the target sequence and to a reference sequence (RPP30) of known copy number, droplet PCR master mix and fluorescent probes specific to the target and reference. For the DUF1220 CON2 locus, the primer sequences are: AGGAATCTGCAGGAGTCTGA (SEQ ID NO:19) and TACGAGGCCAACATTTCAGG (SEQ ID NO:20), and the probe sequence is AGAGGAGGAAGTCCCCCAGG (SEQ ID NO:21). For the reference RPP30 gene (Ribonuclease P protein subunit p30), the primer sequences are GATTTGGACCTGCGAGCG (SEQ ID NO:22) and GCGGCTGTCTCCACAAGT (SEQ ID NO:23), and the probe sequence is TTCTGACCTGAAGGCTCTGCGC (SEQ ID NO:24) (this probe was designed and used with an internal ZEN™ quencher (BIORAD): TTCTGACCT/ZEN/GAAGGCTCTGCGC). Oil droplets are generated using 20 ul PCR mix and 70 ul oil on the QX100 Droplet Generator. These droplets are subjected to a standard thermocycler protocol at an annealing temperature of 56° C., producing over 14,000 independent PCR reactions per sample. Copy number of the target sequence is estimated by comparing the ratio of the target to the reference. Each sample is run in triplicate to confirm results and the copy estimates are merged to produce a final copy count for each sample.

### Example 3

### CON2 IQ Testing

This example demonstrates the use of arrayCGH to assay the copy number of DUF1220 domain clade CON2 in 59 non-Hispanic white individuals with brain size extremes as measured by MRI (the NIMH sample set.). A significant association between IQ and increasing CON2 copy ratio (copy number) in males was found and a similar trend found in females that approached but did not reach significance. Though the trend in females is not as strong, only a small number of females were tested. The results in the following table were found with CON2 in males including an interaction term for age (beta is the slope, the intercept beta value is the global mean IQ in males).

| Males | beta | p-value |
|---|---|---|
| Intercept | 112.7 | <0.01 |
| con2 | 13.8 | 0.011* |
| Age | −0.51 | 0.47 |
| con2*age | −0.96 | 0.036* |

On average, for each 0.1 unit increase in copy ratio WISC full scale IQ increased 1.38 points (p=0.011). This decreases slightly with increasing age (con2*age). Further, 13% of the variation in IQ can be explained by the variation in CON2 copy number (R²=0.13).

This association was verified by assaying 28 (17 males) for CON2 copy number using an independent method, ddPCR (Example 2). The correlation between CON2 values obtained by ddPCR and arrayCGH is 0.75. The association remains in these 17 males and not in females. There were not enough samples to explore age effects. In these 17 males, each copy increase of CON2 is associated with an on average IQ increase of 3.3 (SE=1.5) points p=0.045. Importantly variation of CON2 copy number (dosage) explains 22% of the variation in IQ. The ddPCR-generated values represent a 10% gain in the IQ association compared to results obtained by arrayCGH in the same sample, and represents a full order of magnitude over what has been published in prior studies attempting to link various genes with IQ.

### Example 4

### CON2 IQ Testing

This example demonstrates testing for CON2 copy number correlation with IQ in a different human population: 55 individuals with cognitive measures from the CHDS cohort, confirming an IQ association with CON2 copy number. The following table presents Pearson correlations between DUF1220 domain clades (sub-types) and measures of cognitive ability in a sub-sample of the CHDS cohort (n=54-56).

| Cognitive Measure | CON1 | CON2 | CON3 | HLS1 | HLS2 | HLS3 | PDE4dip_exon |
|---|---|---|---|---|---|---|---|
| WISC_R Verbal IQ (8-9 yrs) | .01 | .23* | .20 | .20 | .22 | .22 | .11 |
| WISC_R Performance IQ (8-9 yrs) | .00 | .09 | .04 | .07 | .07 | .03 | −.08 |
| WISC_R Total IQ (8-9 yrs) | .01 | .18 | .14 | .16 | .17 | .15 | .02 |
| PAT¹ Reading (10 yrs) | .02 | .11 | .03 | .04 | .06 | .07 | −.15 |
| PAT¹ Mathematics (11 yrs) | .23* | .32** | .13 | .22 | .18 | .21 | .01 |
| TOSCA² (13 yrs) | .08 | .16 | .02 | .03 | .03 | .06 | −.08 |

*p < .10
**p < .05
¹Progression Achievement Test
²WISC Verbal IQ and Math Apptitude are the strongest associated. (In the NIMH group it was Total WISC IQ.)

1-20. (canceled)

21. A method to select an individual who is predicted to have a low or high intelligence quotient (IQ) comprising:
a) obtaining a biological sample from an individual, said sample comprising genomic DNA from the individual;
b) detecting in said genomic DNA the copy number of CON2 subtype of DUF1220 domain;
c) correlating the CON2 subtype DUF1220 copy number with an increased likelihood that the individual will have a low, intermediate or high intelligence quotient (IQ).

22. The method of claim 21, wherein the correlating step comprises comparing the copy number of the CON2 subtype of DUF1220 in the biological sample to a control copy number of the CON2 subtype of DUF1220 selected from the group consisting of:
i) a copy number range of the CON2 subtype DUF1220 that has been correlated with IQ less than 100;
ii) a copy number range of the CON2 subtype DUF1220 that has been correlated with IQ greater than or equal to 140; and
iii) a copy number range of the CON2 subtype DUF1220 that has been correlated with IQ between 100 and 140.

23. The method of claim 21, wherein the correlating step further comprises predicting the individual to have a low IQ, if the copy number of the CON2 subtype of DUF1220 in the individual's biological sample is statistically similar to or less than the copy number of the CON2 subtype of DUF1220 that has been correlated with IQ less than 100.

24. The method of claim 21, wherein the correlating step further comprises predicting the individual to have a high IQ, if the copy number of the CON2 subtype of DUF1220 in the individual's biological sample is greater than the copy number of the CON2 subtype of DUF1220 that has been correlated with IQ greater than 140.

25. The method of claim 21, wherein the correlating step further comprises predicting the individual to have an intermediate IQ, if the copy number of the CON2 subtype of DUF1220 in the individual's biological sample is in the same range as the copy number of the CON2 subtype of DUF1220 that has been correlated with IQ between 100 and 140.

26. The method of claim 21, wherein the step of detecting is performed by Droplet Digital PCR quantification of the copy number of the CON2 subtype of DUF1220.

27. The method of claim 26, wherein the step of detecting is conducted using at least one polynucleotide probe selected from the group consisting of:
(SEQ ID NO: 19)
AGGAATCTGCAGGAGTCTGA;
(SEQ ID NO: 20)
TACGAGGCCAACATTTCAGG;
(SEQ ID NO: 21)
AGAGGAGGAAGTCCCCCAGG
(SEQ ID NO: 22)
GATTTGGACCTGCGAGCG;
(SEQ ID NO: 23)
GCGGCTGTCTCCACAAGT;
and
(SEQ ID NO: 24)
TTCTGACCTGAAGGCTCTGCGC.

28. The method of claim 26, wherein an individual having 33 or more diploid copies of the CON2 subtype of the DUF1220 domain in the biological sample is predicted to have an IQ greater than 140.

29. The method of claim 26, wherein an individual having 26 or fewer diploid copies of the CON2 subtype DUF1220 domain in the biological sample is predicted to have an IQ less than 100.

30. The method of claim 26, wherein an individual having between 26 and 33 diploid copies of the CON2 subtype of the DUF1220 domain in the biological sample is predicted to have an IQ between 100 and 140.

31. The method of claim 26, wherein the copy numbers of the CON2 subtype of DUF1220 correlated with an IQ less than 100, an IQ between 100 and 140, and an IQ greater than or equal to 140 are predetermined control gene copy numbers.

32. The method of claim 21, wherein the step of detecting is performed using arrayCGH.

33. The method of claim 21, wherein the step of detecting is performed by measuring DNA sequence read depth.

34. A method of enhancing cognitive function in an individual comprising administering an effective amount of DUF1220 protein to the individual.

35. A method of enhancing or increasing the expression, amount, dosage, effect or activity of a DUF1220 domain protein in an individual comprising administering an agent selected from the group consisting of:
a) a nucleic acid that increases the production of a DUF1220 protein.
b) a protein that increases the production of a DUF1220 protein,
c) a naturally-occurring cognate ligand of a DUFF1220 protein,
d) a DUF1220 protein,
e) a peptidomimetic of a DUF1220 protein, and
f) a small molecule that increases the production of a DUF1220 protein.

36. A method to select an individual who is predicted to develop macrocephaly or microcephaly comprising:
a) obtaining a biological sample from an individual, said sample comprising genomic DNA from the individual;
b) detecting in the biological sample the copy number of the DUF1220 domain;
c) detecting the copy number of genes in the genomic DNA immediately flanking genes encoding DUF1220 domains.
d) comparing the copy number of the DUF1220 domain in the biological sample to a control copy number of the DUF1220 domain;
e) comparing the copy number of the genes in the genomic DNA immediately flanking genes encoding DUF1220 domains in the biological sample to a control copy number of the genes in the genomic DNA immediately flanking genes encoding DUF1220 domains;
f) selecting the individual as being predicted to develop macrocephaly, if the DUF1220 copy number in the individual's genomic DNA and the copy number of the genes in the genomic DNA immediately flanking genes encoding DUF1220 domains are statistically similar to or greater than the control DUF1220 copy number and the control copy number of the genes in the genomic DNA immediately flanking genes encoding DUF1220 domains that have been correlated with macrocephaly; and,
g) selecting the individual as being predicted to develop microcephaly, if DUF1220 copy number in the individual's genomic DNA and the copy number of the genes in the genomic DNA immediately flanking genes encoding DUF1220 domains are statistically similar to or less than the control DUF1220 copy number and the control number of the genes in the genomic DNA immediately flanking genes encoding DUF1220 domains that have been correlated with microcephaly.

37. The method of claim 36, wherein the step of detecting is performed by arrayCGH quantification of the copy number of the DUF1220 domain and flanking genes in the genomic DNA from the patient.

38. The method of claim 36, wherein the detecting steps are performed by Droplet Digital PCR quantification of the copy number of the DUF1220 domain and flanking genes in the genomic DNA from the patient.

39. The method of claim 36, wherein the detecting steps are performed by measuring DNA sequence read depth on the genomic DNA from the individual.

40. A kit for detecting DUF1220 domain gene copy numbers in a biological sample comprising at least one of:
(a) a means for detecting in a biological sample a DNA copy number of DUF1220 domain;
(b) means for determining a control value selected from:
(i) a control sample for detecting low DUF1220 DNA copy levels;
(ii) a control sample for detecting high DUF1220 DNA copy levels;
(iii) information containing a predetermined control DNA copy number of the DUF1220 domain.
(c) an internal standard control gene with a known copy number to which DUF1220 copy number measurements can be compared.
